(19) United States
(12) Patent Application Publication
(10) Pub. No.: US 2010/0016741 A1
(43) Pub. Date: Jan. 21, 2010

(54) Heart rate monitor

(76) Inventors: John Mix, Brentwood, CA (US); Roar Viala, Banyuls Surmer (NO)

Correspondence Address: James A. Gavney Jr.;JAG Patent Services LLC, Suite 21, 1901 Old Middlefield Way, Mountain View, CA, 94043, US

(21) Appl. No.: 12/459,494

(22) Filed: Jul. 2, 2009

Related U.S. Application Data
(60) Provisional application No. 61/135,491, filed on Jul. 21, 2008.

(51) Int. Cl. A61B 5/024

(52) U.S. Cl. 600/508

(57) ABSTRACT

A waterproof heart rate monitor device, method, and system are disclosed that transmit heart rate information to a user. In operation one or more infrared sensor sits against skin of a user to measure his or her heart rate. An internal computer, or micro-processor, that is coupled to one or more sensors then calculates the number of beats/minute (b/m) that a user's heart is beating and generates output signals to an output unit such as an ear plug or via bone conduction transducer to provide a user an audio representation of his or her heart rate.

### RELATED APPLICATION

This Application claims priority under 35 U.S.C. §119(e) from the U.S. Provisional Patent Application Ser. No. 61/135,491, filed on Jul. 21, 2008 and titled “HEART RATE MONITOR.” The co-pending U.S. Provisional Patent Application Ser. No. 61/135,491, filed on Jul. 21, 2008 and titled “HEART RATE MONITOR” is hereby incorporated by reference.

### FIELD OF THE INVENTION

This invention relates generally to athletic training devices. More specifically, this invention relates to electronic devices used to measure heart rates and aid athletes while training.

### BACKGROUND OF THE INVENTION

Heart-rate is the only accurate measurement of your intensity or your exertion level, and a heart rate monitor is the easiest and most precise way to continuously measure your heart rate. Heart rate monitors, or HRM's, allow you to analyze workouts and races. HRM's can show you when you're dehydrating, or running out of nutrition, or not recovered from a previous day's workout.

Current versions of HRM's consist of a watch worn on your wrist and a transmitter that you wear against your skin and around your chest. The transmitter picks up the signals of your heart and sends them wirelessly to the watch you wear on your wrist. This setup may be good for running and dryland training, but it is quite awkward and inconvenient for training in the water. The chest strap is cumbersome for swimmers, often loosening or falling off, and provides an inaccurate heart rate. Additionally, if a swimmer is using current HRM's, he or she can only read his or her heart rate while stopped. Thus, the swimmer will receive skewed results during stoppage rather than immediate and continuous feedback while swimming. As a result, current HRM's do not provide a precise understanding of training heart rate during aquatic fitness exercises such as swimming.

### SUMMARY OF THE INVENTION

It is therefore an object of the present invention to provide a waterproof heart rate monitor device to allow a user to measure his or her heart rate underwater through changes in light via the user's skin and to hear underwater audio signals reporting his or her heart rate via an ear plug that are generated by transcutaneous bone conduction.

A further object of the invention is to provide a heart rate monitor that can be completely and compactly secured to a user's goggle strap or other means of support at or near the user's temple via a small clip.

Additionally, an object of the present invention is to enable the user to output his or her workout data to a computer and generate an electronic and paper printout of his or her workout based on the heart rate monitor's recorded data.

The heart rate monitor of the present invention has been developed with the needs of swimmers in mind. The heart rate monitor has a waterproof design that changes both how the heart rate is sensed or measured and how the measured heart rate is transmitted or communicated to the user. All functions of the heart rate monitor are preferably integrated into a unit that clips on to a goggle strap or other support and rests on or near the temple of the user.

In operation, an infrared sensor sits against skin near or at the user's temple and picks up the heart rate. One or more sensors measure the changes in light due to blood flow in the skin. A internal computer, or micro-processor, that is coupled to the one or more sensors then calculates the number of beats/minute (b/m) that the heart is beating. Additionally, a second sensor measures background noise, and the micro-processor generates a correction factor for calculating the heart rate based on the background noise. The micro-processor then generates output signals to an output means. The output means, such as an ear plug, then conveys audible signals to the user via bone conduction, thereby providing the user with an indication of his or her heart rate.

Humans normally hear through air conduction, but because there is no air underneath the water, bone conduction provides the clearest sound quality possible. In using bone conduction, the sound vibrations are communicated through the temple bone to the inner ear where sound is transferred. By simply turning the heart rate monitor on, the user can sense his or her heart rate, which is automatically communicated during the swim.

### BRIEF DESCRIPTION OF THE DRAWINGS

FIG. 1 shows a schematic representation of a heart rate monitor device, in accordance with the embodiments of the invention.

FIG. 2 shows a block-flow diagram outlining steps for generating an audio representation of a heart rate, in accordance with the method of the invention.

FIG. 3 shows a heart rate monitor system, in accordance with the embodiments of the invention.

FIG. 4 shows a representation of an output unit for communicating an audio representation of heart rate information to a user via bone conduction, in accordance with the embodiments of the invention.

FIG. 5 shows a heart rate monitor with output unit for communicating heart rate information to a user via bone conduction and a infrared sensor unit for measuring a user's heart rate through the user's ear, in accedence with a preferred embodiment of the invention.

### DETAILED DESCRIPTION OF THE INVENTION

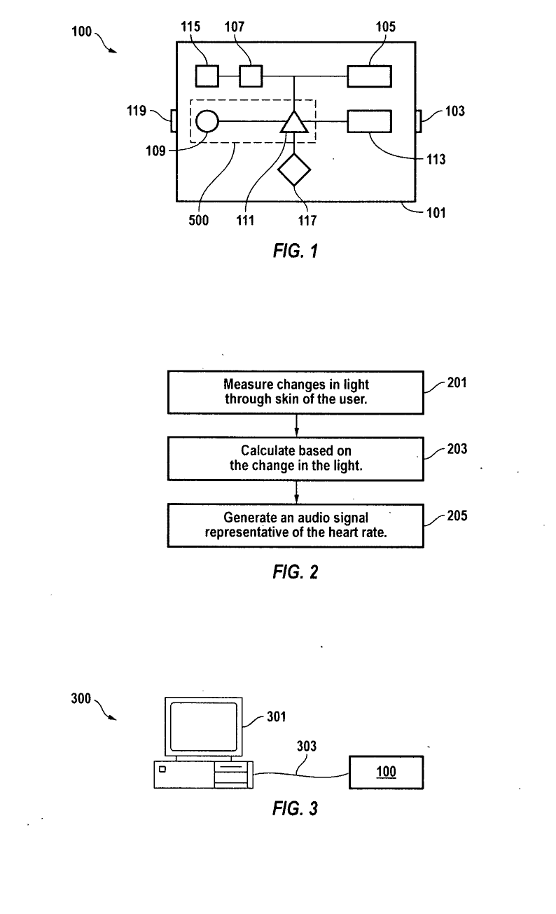

Referring to FIG. 1, heart rate monitor 100 includes a housing 101, that can have any suitable dimensions. The heart rate monitor 100 preferably include an attachments means for attaching the heart rate monitor 101 or a portion thereof a user's head and/or a pair of goggles (not shown). The attachment means 103 is a clip, a snap, a strap or any other suitable attachment means. The heart rate monitor 100 includes a user interface 105 that controls modes of operation, such as turning the heart rate monitor on and off, selecting pre-set time-periods that heart rate is reported through an output unit 113 and changing the volume of the output means 113.

The output unit 113 is configured to produce an audio representation of a user's heart rate. The output unit 113 is preferably an ear phone or a bone conduction transducer that transmits audio signals through a bony portion of the user's head. While people usually hear sound through air conduction, this is generally not suitable in aquatic environments. Using a bone conduction transducer allows the heart rate monitor 100 to produce vibrations that are transmitted through the bony portion of the user's head and produce audible sound within the inner ear or ears of the user.

Still referring to FIG. 1, the heart rate monitor further includes a micro-processor 107 that is programmed with firm-ware that calculates heart rate based on changes in the light detected by a senor unit 500 with one or more light sensors 109 and 111 and then generates output signals that are played by the output unit 113.

In accordance with the embodiments of the invention the senor unit 500 includes a first infrared sensor 109 that measures a heart rate based in blood flow through a portion of a user's skin and a second infrared sensor 111 that measures background noise from environmental factor including, but not limited to, ultraviolet light. In accordance with these embodiments of the invention the micro-processor 107 and the associate firm-ware calculate a correction factor for calculating the heart rate and generates corrected output signals that are played by the output means 113. The one or more sensors 109 and 111 are built-in to the housing 101 of the heart monitor 100 or are housed in a separate sensor unit, such as the sensor unit 505 (FIG. 5), which measures a user's heart rate by detecting changes in blood flow through an ear lobe of the user.

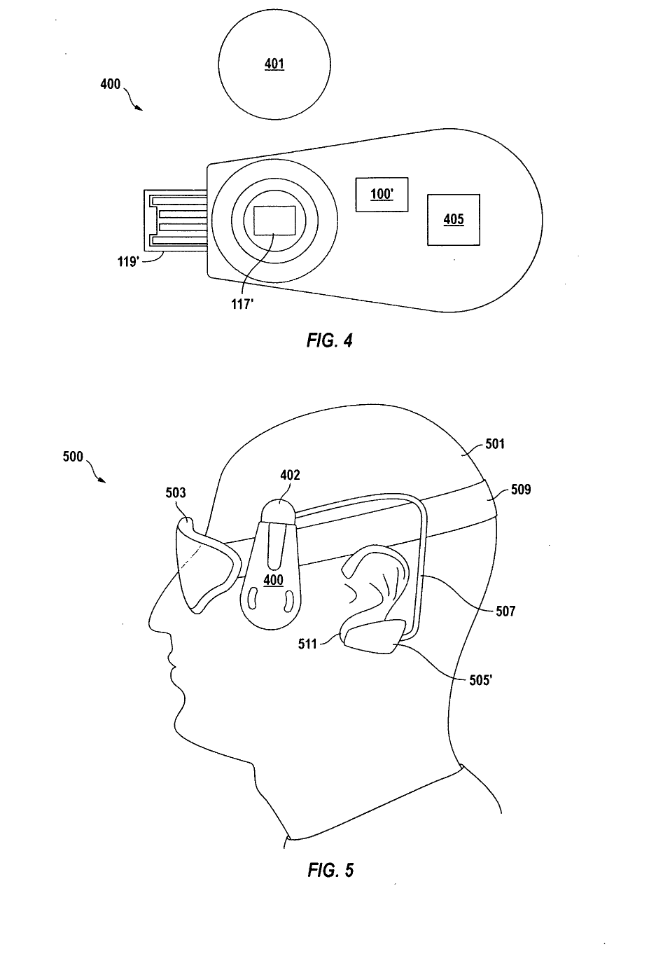

Still referring to FIG. 1, the heart rate monitor 100 preferably includes a memory unit 115 that stores history data of the user's heart rate during a workout that can be downloaded to a computer 301 (FIG. 3) through, for example, USB plug 119, to generate a graphical representation of the user's heart rate during the workout. The memory unit 115 includes a permanent memory source, a removable memory source (such a secure memory disk) or a combination thereof. The heart rate monitor 100 also includes a power unit 117, that preferably includes an internal permanent or removable and rechargeable battery 117, such as a lithium-ion battery. The power unit 117 is, for example, rechargeable through the USB plug 119 and/or through an adaptor (not shown) that plugs into a wall outlet.

FIG. 2 indicates a block-flow diagram 200 outlining steps for generating an audio representation of a heart rate, in accordance with the method of the invention. In the step 201, changes in light are measured through a user's skin. For example, changes in light are measured using an infrared sensor unit 500′ (FIG. 5) that is coupled to or clipped on to a user's ear. After changes in light are measured through a user's skin in the step 201, in the step 203 a heart rate is calculated using input signals from the senor unit 500′ and the micro-processor 107 (FIG. 1) based on the changes in the light measured by the sensor unit 500′. After the heart rate is calculated in the step 203, the micro-processor 107 generates output signals that are transmitted to the output unit 113 where the output signals are played in the step 205 to generate an audio signal representative of the heart rate.

Referring now to FIG. 3, a system in accordance with the embodiments of the invention includes a heart rate monitor 100, a computer 301 and a coupling means 303 for connecting the heart rate monitor 100 to the computer 301. The coupling means 303 is any suitable chord, such as a USB chord or an Ethernet Chord, or a wireless device, such as an infrared transducer or a radio transducer. For simplicity of the description, a heart rate monitor in the system 300 is represented by the heart rate monitor 100, such as shown in FIG. 1. It is, however, understood that the system 300 of the present invention in further embodiments of the invention includes a heart rate monitor with components 101′, 505′ and 507, such as represented in, and described with reference to in FIGS. 4 and 5.

In operation the heart rate minor 100 is connected to the computer 301 through the coupling means. Workout history data that has been collected by the heart rate monitor and in the memory unit 115 is downloaded to the computer 301. The computer 301 is preferably programed with the appropriate driver to read workout history data from the heart rate monitor 100 and also is preferably programmed with software, such the computer 301 is capable of generating a graphical representation of the workout history data. It will be clear to one skilled in the art that the workout history data can be manipulated in any number of ways to generate a number of different geographical representations of the workout heart rate data to provide insight into the user's workout and the users's workout performance.

FIG. 4 shows a representation of an output unit 400 for communicating an audio representation of heart rate information to a user via bone conduction, in accordance with the embodiments of the invention. The output unit 400 includes a bone conduction transducer 405 for transmitting output signal to a bony portion of a users head, such as described above. Further details of using bone conduction to transmits audio signals are provided in U.S. patent application Ser. No. 10/830,390, filed Mar. 18, 2004 and titled “UNDER WATER ENTERTAINMENT SYSTEM,” the contents of which are hereby incorporated by reference. The output unit 400 also includes a power unit 117′ that is preferably a rechargeable battery, such as described with reference to 117 in FIG. 1. The power unit 117′ is, for example, rechargeable through a USB plug 119′ that is configured to plug into a USB port on a computer 301 (FIG. 3). The power unit 117′ is preferably housed within the output unit 400 and sealed with a water proof cap 401. The output unit 400 includes all the necessary components and circuitry 100′ to receive input signals for a sensor unit 505′ (FIG. 5), process input signals, generate output signals and transmit the output signals to output unit 400 to generate an audio representation of a user's heart rate, such as described above. The necessary components and circuitry includes, but is not limited to, a user interface, a microprocessor, a memory unit, related contacts and connections.

FIG. 5 shows a heart rate monitor with output unit 400 for communicating heart rate information to a user via bone conduction and an infrared sensor unit 505′ for measuring a user's heart rate through the user's ear 511, in accordance with a preferred embodiment of the invention. The output unit 400 includes a clip (not shown) for attaching to a strap of a pair of goggles 503 and a cap structure for covering the USB plug 119′ (FIG. 4) While the pair of goggles is attached to the user's head, the output unit 400 is placed with the bone conduction transducer 405 against a bony portion of the user's head and transmit audio signals via bone conduction. The infrared sensor unit 505′ is configured to clip on to the ear lobe of the user and detect changes in blood flow through the ear lobe to determine the user's heart rate. The infrared sensor unit 505′ preferably includes a first sensor and a second sensor, such as the first sensor 109 and the second sensor 111 (FIG. 1) to provide a more accurate reading of the user's heart rate. The output unit 400, the infrared sensor unit 505′ or a combination thereof include all the necessary components and circuitry for processing input signals from the sensor unit 505′ and generate output signals to the output unit 400 to generate an audio representation of the user's heart rate.

The present invention has been described in terms of specific embodiments incorporating details to facilitate the understanding of the principles of construction and operation of the invention. For example, a heart rate monitor can include a sensor unit for detecting changes in blood flow and can be configured to attach skin on any number of areas of a user's body. Further, the heart rate monitor can include a number of output units that include bone conduction transducers, ear phones, ear plugs and combinations thereof: As such, references herein to specific embodiments and details thereof are not intended to limit the scope of the claims appended hereto. It will be apparent to those skilled in the art that modifications can be made in the embodiments chosen for illustration without departing from the spirit and scope of the invention.

What is claimed is:

1. A device comprising:
a) a light sensor for measuring changes in light due to blood flow in the skin;
b) a micro-processor for calculating heart rate based on changes in the light detected by the infrared sensor and generating output signals; and
c) output means for converting the outputs to audio signals representative of the heart rate.

2. The device of claim 1, further comprising means for positioning the infrared sensor at or near the temple of the user.

3. The device of claim 2, wherein the means for positioning the infrared sensor at or near the temple of the user comprises a clip for attaching the sensor to a strap of a goggle or other support.

4. The device of claim 1, wherein the output means comprises a bone conduction transducer for transmitting audio signals through a portion of the user's head.

5. The device of claim 4, wherein the output means comprises an ear plug.

6. The device of claim 1, further comprising a second infrared sensor for measuring background noise and wherein the micro-processor generates a correction factor for calculating the heart rate based on the background noise.

7. The device of claim 1, wherein the monitor can automatically sense and measure the user's heart rate by simply having the user turn on the waterproof heart monitor during swimming.

8. The device of claim 1, further comprising a user interface for controlling modes of operation.

9. The device of claim 1, wherein the monitor is powered by an internal lithium-ion rechargeable battery and further comprising a USB port for recharging the battery.

10. The device of claim 1, wherein the monitor's battery is recharged through a USB port that can be plugged into a personal computer or wall adaptor.

11. The device of claim 1, further comprising a light sensor/memory unit for storing the history of the user's heart rate during workouts.

12. A method comprising: a) measuring changes in light through the skin of the user; b) calculating a heart rate of the user based on the change in the light detected by the light sensor; and c) generating an audio signal representative of the heart rate.

13. The method of claim 12 wherein measuring change in light through the user's skin comprises placing an infrared sensor at or near the user's skin.

14. The method of claim 12, wherein calculating the heart rate comprises measuring background noise and subtracting the background noise from light measured through the skin.

15. The method of claim 14, wherein the second infrared sensor measures background noise, and the micro-processor generates a correction factor for calculating the heart rate based on background noise.

16. The method of claim 12, further comprising generating audio signals through a transducer attached to the ear or temple via bone conduction.

17. The method of claim 12, wherein the bone conduction transducer communicates the heart rate to the user through vibrations to the user's temple bone and to the user's inner ear where sounds are transferred.

18. A system comprising:
a) a heart rate monitor for measuring a user's heart rate comprising a light sensor, micro-processor, and audio output; and
b) means for generating a graphical representation of the user's heart rate.

19. The system of claim 18, wherein the heart rate monitor further comprises an infrared sensor for measuring changes in light due to blood flow in the skin, a clip to attach the heart rate monitor to the user's goggle or other means of support, a second sensor for measuring background noise, and a micro-processor for calculating heart rate and for generating a correction factor for calculating the heart rate based on background noise.

20. The system of claim 18, comprising a computer to process workout data from the heart monitor and to generate a graphical representation of the user's heart rate based on such data.
